(19) United States
(12) Patent Application Publication
(10) Pub. No.: US 2022/0204590 A1
(43) Pub. Date: Jun. 30, 2022

(54) Antibody-based blockage of SARS-CoV-2-specific furin cleavage site

(71) Applicants: Weiwei Li, Woodbury, NY (US); Jessica Mliu Li, Woodbury, NY (US); William Lei Lee, Mineola, NY (US)

(72) Inventors: Weiwei Li, Woodbury, NY (US); Jessica Mliu Li, Woodbury, NY (US); William Lei Lee, Mineola, NY (US)

(21) Appl. No.: 17/137,707

(22) Filed: Dec. 30, 2020

(51) Int. Cl. C07K 16/10

(52) U.S. Cl. CPC C07K 16/10; A61K 2039/505

(57) ABSTRACT

The present invention provides an antibody and its use method for immunologically blocking furin cleavage site on the S1/S2 boundary of SARS-CoV-2 spike protein that are in purified form or from a biological sample to prevent the SARS-CoV-2 spike protein cleavage caused by furin and furin-related proteases.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

### Field of the Invention

This invention is related to an antibody and its use method for immunologically blocking furin cleavage site on the S1/S2 boundary of SARS-CoV-2 spike protein that is in purified form or from a biological sample to prevent the SARS-CoV-2 spike protein cleavage from furin and furin-related proteases.

### Description of the Related Art

The recent COVID-19 pandemic is caused by SARS-CoV-2, a new member of the same coronavirus family that caused SARS and MERS. So far, there are no effective drugs that can be used for prevention and treatment of COVID-19. It has been observed that in the same exposure environment to SARS-CoV-2, some people become infected while others do not. Those infected with SARS-CoV-2 can either display severe illness or be asymptomatic/mildly symptomatic.

It was found that the SARS-CoV-2 spike (S) glycoprotein harbors a furin cleavage site at the boundary between the S₁/S₂ subunits, which could be cleaved by furin and/or furin-like PCs secreted from host cells and bacteria in the airway epithelium. Unlike SARS-CoV, cell entry of SARS-CoV-2 needs to be pre-activated by furin and/or furin-like PCs, reducing its dependence on target cell proteases for entry. The cleavage activation of S-protein is well demonstrated to be essential for SARS-CoV-2 spike-mediated viral binding to the host ACE2 receptor, cell-cell fusion, and viral entry into human lung cells. It was also observed that other viruses containing a furin cleavage site, such as H5N1, displayed increased replicates and developed higher pathogenicity.

The complete SARS-CoV-2 furin cleavage site has been characterized as a 20 amino acid motif that is corresponding to the amino acid sequence ^{A}672-^{S}691 of SARS-CoV-2 spike protein (QOS45029.1), with one core region SPRRAR|SV (eight amino acids, ^{S}680-^{V}687) and two flanking solvent accessible regions (eight amino acids, ^{A}672-^{N}679, and four amino acids, ^{A}688-^{S}691). The core region is very unique as its ^{R}683 and ^{A}684 positions are positively charged (Arg) and hydrophobic (Ala) residues, respectively, which allow this site to not only be cleaved by serine protease furin or furin-like PCs, but also permit cleavage efficiency to be facilitated by other serine proteases targeting mono- and dibasic amino acid sites such as matriptase, kallikrein 1 (KLK1), human airway trypsin (HAT), and TMPRSS2. Furthermore, a serine at ^{S}680 of the core region could also highly increase the cleavage efficiency, causing increased viral replication, unrestricted organ tropism, and virulence and mortality rate as proven in H5N1 infection studies in mice.

Furin and furin-like PCs, such as PC5/6A and PACE4, are proven to be cleavage region sequence-specific and these PCs exhibit widespread tissue distribution. With the unique furin cleavage site in SARS-CoV-2, such distribution may explain why COVID-19 causes damage in multiple organs. Thus, the importance of blocking SARS-CoV-2 S1/S2 site cleavage caused by furin or facilitating protease activity is emphasized by the fact that furin-based SARS-CoV-2 S1/S2 cleavage increases SARS-CoV-2 entry into cells and its replication, and eventually develops higher pathogenicity and transmission of COVID-19.

The current approaches to block or reduce furin site cleavage of the target proteins are based on the inhibition of furin or furin-like enzymes. Such inhibition is generally achieved by naturally occurring macromolecular protein-based inhibitors such as serpin al-antitrypsin or small molecule chemical inhibitors such as pure peptide, peptide mimetics and nonpeptidic compounds. Currently there are many small molecular inhibitors available for inhibiting activity of proteases including furin and furin-like PCs. These inhibitors, with different action mechanisms are selective for furin and different PCs, which would limit the effect of a furin inhibitor in protecting cleavage of a furin site from furin and different PCs. Furthermore, furin and furin-related PCs are widely distributed in various human tissues. The inhibition of host furin and furin-related PCs could non-specifically cause damage of normal functions of the proteins that need activation by furin or furin-related PCs. There are no methods other than direct furin inhibition available for blockage of SARS-CoV-2 specific furin site cleavage by furin or facilitating proteases. Therefore there is a need to develop such blockage methods.

### BRIEF SUMMARY OF THE INVENTION

The present invention provides an antibody and its use method of this antibody for immunologically blocking furin cleavage site on the S1/S2 boundary of SARS-CoV-2 spike protein to prevent the SARS-CoV-2 spike protein cleavage by furin and facilitating proteases, comprising the steps of:

1) Generating an antibody against an antigen containing SARS-CoV-2 S1/S2 sequences that can be cleaved by furin and facilitating protease;
2) Determining the specificity and sensitivity of the antibody against SARS-CoV-2 spike protein containing S1/S2 boundary furin site;
3) Determining the binding affinity of the antibody to the spike protein containing S1/S2 boundary furin site and a peptide containing SARS-CoV-2 specific furin cleavage site;
4) Determining the cleavage of SARS-CoV-2 specific furin site by purified furin and facilitating proteases in the presence and absence of the antibody;
5) Determining the cleavage of SARS-CoV-2 specific furin site by a biological nasal swab sample in the presence and absence of the antibody.

Thus the invention allows for an antibody and its use method for immunologically blocking furin cleavage site on the S1/S2 boundary of SARS-CoV-2 spike protein to prevent the SARS-CoV-2 spike protein cleavage by furin and furin-related proteases. The invention is based on the finding that the SARS-CoV-2 spike protein contains a specific furin motif on S1/S2 boundary which can be cleaved not only by furin but also by certain proteases. The invention is also based on the finding that an antibody against this furin motif can tightly bind to SARS-CoV-2 spike protein. The invention is further based on the finding that the immunologically blocking furin cleavage site on the S1/S2 boundary of SARS-CoV-2 spike protein can prevent the SARS-CoV-2 spike protein cleavage caused by furin and facilitating proteases. Therefore, the method presented in this invention significantly overcomes the weaknesses existing in the prior technologies and enables SARS-CoV-2 cleavage by furin and facilitating proteases to be blocked efficiently.

### BRIEF DESCRIPTION OF THE DRAWINGS

FIG. 1 shows the furin site blocking antibody (fbAB) generation and its titer of recognizing the antigen containing SARS-CoV-2 specific furin motif using the method of this invention. The experiment was carried out as described in Example 1. Antigen concentration (200 ng/well).

FIG. 2 shows the sensitivity of the fbAB recognizing the antigen containing SARS-CoV-2 specific furin motif using the method of this invention. The experiment was carried out as described in Example 2.

FIG. 3 shows the specificity of the fbAB recognizing the SARS-CoV-2 spike protein containing S1/S2 boundary furin site or a peptide containing SARS-CoV-2 specific furin motif using the method of this invention. The experiment was carried out as described in Example 3. A: SARS-CoV-2 protein containing S1/S2 boundary furin site; B: Peptide containing SARS-CoV-2 specific furin motif; C: SARS-CoV-2 S1 RBD protein lacking S1/S2 boundary furin site.

FIG. 4 shows the immunoprecipitation of the SARS-CoV-2 protein containing S1/S2 boundary furin site by the fbAB using the method of this invention. The experiment was carried out as described in Example 4. A: SARS-CoV-2 protein containing S1/S2 boundary furin site; B: Peptide containing SARS-CoV-2 specific furin motif.

FIG. 5 shows the fbAB blockage of the SARS-CoV-2 furin motif cleavage caused by furin using the method of this invention. The experiment was carried out as described in Example 5. Furin concentration: 2-4 units/well; fbAB and Spike protein neutralization antibody (SnAB) concentration: 200 ng/well.

FIG. 6 shows the fbAB blockage of the SARS-CoV-2 furin motif cleavage caused by facilitating protease trypsin using the method of this invention. The experiment was carried out as described in Example 6. Trypsin concentration: 10-20 ng/well; fbAB concentration: 200 ng/well.

FIG. 7 shows the fbAB blockage of the SARS-CoV-2 furin motif cleavage caused by human nasal swab sample using the method of this invention. The experiment was carried out as described in Example 7. Human nasal swab sample: released into 300 μl of furin assay buffer and 20-30 μl of sample solution was used for assay; fbAB concentration: 200 ng/well.

FIG. 8 shows reduction of ACE2 binding to SARS-CoV-2 spike protein by fbAB blocking the SARS-CoV-2 furin motif at different concentrations using the method of this invention. The experiment was carried out as described in Example 8. Coated SARS-CoV-2 spike protein concentration: 50 ng/well. ACE2 concentration: 100 ng/well.

### DETAILED DESCRIPTION OF THE INVENTION

The present invention provides an antibody fbAB and its use method for immunologically blocking furin cleavage site on the S1/S2 boundary of SARS-CoV-2 spike protein to prevent the SARS-CoV-2 spike protein cleavage caused by furin and facilitating proteases. This antibody binding to the SDARS-Cov-2 specific furin site is particularly useful for blocking SARS-CoV-2 cleavage by furin or facilitated proteases.

According to the method of this invention, the antigens used for fbAB generation could be a synthesized peptide containing a certain length of SARS-CoV-2 S1/S2 boundary sequence including furin cleavage motif. The peptide can be consisted of minimal 8 amino acids and maximal 100 amino acids in the length, which contains at least a SARS-CoV-2 specific core region (^{R}682-^{V}687) of furin cleavage motif, or a full SARS-CoV-2 specific furin cleavage motif (20 amino acids), which is corresponding to the amino acid sequence ^{A}672_s₆₉₁ of SARS-CoV-2 spike protein (QOS45029.1). The peptide may contain multiple SARS-CoV-2 specific core regions of furin cleavage motif, or multiple full SARS-CoV-2 specific furin cleavage regions in certain embodiments. The peptide antigen can be synthesized through a commercially available service or generated by a solid phase peptide synthesis method using Fmoc-chemistry, which allows for synthesizing of peptides with a length up to 50 amino-acids. In specific embodiments, a longer peptide can be synthesized by fragment synthesis and chemical ligation technologies, which allows for synthesizing peptides with a length up to 150 amino-acids. To increase antibody titers and induce a prolonged response with accompanying memory, the synthesized peptide can be conjugated with adjuvants such as KLH and BSA. KLH may be modified with 3-sulfo-N-hydroxysuccinimide ester sodium salt before conjugation. The conjugates of KLH-antigen can be identified by ultraviolet spectrophotometry.

According to the method of this invention, the antibodies can be purified polyclonal antibodies or monoclonal antibodies in different types, preferably, in IgG form. Polyclonal antibodies can be generated from animals that have been exposed to the antigen. Standard protocol for polyclonal antibody generation is publicly available. The animals often used for polyclonal antibody generation may include rabbit, mouse, chickens, goats, rat, guinea pigs, hamsters, sheep, and horses. Rabbit should be the most commonly used animal for polyclonal antibody production. (2) Injection of antigen/adjuvant can be injected into animals. Injections of the antigen are given in multiple sites to stimulate the best immunity. When using rabbit, the rabbits are boosted at 3 to 4 week intervals until peak antibody titers are reached (6-8 re-immunizations). Blood is collected and clotted. The clotted blood is then refrigerated for 24 hours before the serum is decanted and clarified by centrifugation. Monoclonal antibodies can be generated with the hybridoma technology that is well known in the art. The antigen is first used for immunizing hosts such as mouse, rat, and rabbit. The antibody-generating cells in the immunized animals are then isolated and fused with myeloma cells to form hybridoma cells. The monoclonal antibodies can be generated and purified from different clones of the hybridoma cells. Monoclonal antibodies may also be produced with other technologies such as using an expressing nucleic acid cloned from a hybridoma or using phage display technology. The produced antibody can refer to different forms such as chimeric antibodies, humanized antibodies, single domain nanobodies, F(ab′)₂ fragments, Fab fragments, Fv fragments, and sFv fragments.

The generated fbAB can be tested for its titers against the antigen in order to measure how much antibody the animal has produced that recognizes the epitope. For titer testing, the antigen is bound to solid-phase carrier by using appropriate buffers. The solid-phase carrier includes but is not limited to polystyrene plastic, glass, silica and metal. The carrier could be in various sizes and forms including, but not limited to beads in size of 10 nm-100 um, strip with 8-wells-12-wells, microplate with 6 wells-1516 wells, microscopic slide with or without wells, and microarray slide with or without wells. Preferably, the plastic microplate or strips are more suitable to be used in the method of this invention, as these carriers are easily handled in a rapid and high throughput format. The appropriate binding buffers include but are not limited to bicarbonate buffer, phosphate buffer, glycine/NaOH buffer, Tris-sodium buffer and HEPES-sodium buffer. The microplate or strip is incubated at 37° C. with or without humidity for 90 min. The beads are incubated at 37° C. with or without humidity for 90 min. The antigen amount to be bound can be from 10 to 400 ng, preferably 200 ng. A 200 ng of antigen amount would ensure the sufficient antigen amount to be detectable. The antibody can be prepared in different dilution ratio from 1:100 to 1:500,000, preferably 1:1000 to 1:250,000. The antibody amount bound to the antigen can be detected with a secondary antibody conjugated with a signal moiety and specific to the species of the generated antibody. The antibody titer can be quantified by optical absorbance or fluorescence measurement of the signal intensity and expressed as the reciprocal value of the last dilution that gives a signal above the background levels.

The generated fbAB can be tested with a direct ELISA method for its sensitivity and specificity in recognizing the SARS-CoV-2 spike protein containing S1/S2 boundary furin site or a peptide containing SARS-CoV-2 specific furin motif. To test sensitivity, the SARS-CoV-2 protein containing furin site and/or peptide containing SARS-CoV-2 specific furin motif is bound to a solid-phase carrier such as a 96-well plate at different concentrations. The sensitivity can be expressed as limit of detection, the lowest protein/peptide concentration to be reliably distinguished from blank background level. To test specificity, the SARS-CoV-2 protein with or without furin site and/or peptide containing or not containing SARS-CoV-2 specific furin motif is bound to the wells of 96-well plate. The specificity can be expressed as signal intensity ratio to discriminate between SARS-CoV-2 protein containing furin site and the SARS-CoV-2 protein without furin site.

According to the method of the invention, the generated fbAB can be further tested for its binding capacity in forming complex with SARS-CoV-2 protein containing furin site. For this test, the antibody can be coated on a solid-phase carrier such as plastic plate wells or magnetic beads. The antibody coating methods are well known in the art. If plastic plate wells are used, the antibody can be directly bound to the wells by using protein binding buffer such as NaCO3 buffer or phosphate balance solution or bound to the wells pre-coated with protein A/G, the IgG affinity protein. If the magnetic beads are used, the beads should be coated with protein A/G before applying the antibody to the beads. The different amount of SARS-CoV-2 protein containing furin site is then added into the wells bound with the antibody. The bound protein can be detected with an anti-SARS-CoV-2 spike antibody and the bound amount of the protein can be quantified by using a secondary antibody conjugated with a signal moiety followed by signal amplification and optical absorbance or fluorescence measurement of the signal intensity. Preferable, the SARS-CoV-2 protein containing furin site can be pre-labeled with the affinity moiety such as biotin and polyhistidine so that the proteins can be directly quantified through binding pairs of affinity moieties-binding partners conjugated with signal moieties. For example the binding pair consists of biotin and streptavidin, polyhistidine and nickel. The binding capacity can be expressed as the maximum amount of the protein bound to the antibody at the given protein concentration conditions.

According to the method of the invention, blockage of SARS-CoV-2 specific furin motif by the generated fbAB can be performed in the presence or absence of furin, furin-like PCs and facilitating proteases. The furin, furin-like PCs and facilitated proteases can be recombinant proteins with enzyme activity. These PCs or facilitated proteases are capable of cleaving the SARS-Cov-2 specific furin site substrate. These PCs and facilitated proteases may include but are not limited to proprotein convertases such as furin, PC1/3, PC2, PACE4, PC5/6A, PC7, SKI-1/S1P and PC9; type II transmembrane serine proteases such as human air trypsin (HAT), TMPRSS2, TMPRSS4, TMPRSS11A, matriptase; cysteine proteases such as cathepsin A, cathepsin B and cathepsin L; thrombin-like protease such as plasmin, thrombin, and tissue activating plasminogen. To test the blockage of the SARS-CoV-2 specific furin motif by the generated fbAB, the SARS-CoV-2 spike protein containing S1/S2 boundary furin site can be tagged with an affinity moiety such as biotin or polyhistidine at N-terminal or C-terminal. The tagged protein is bound to the plate wells coated with a binding partner of the affinity moiety such as streptavidin for biotin and nickel for polyhistidine, respectively. The fbAB is added to the bound protein for an appropriate time followed by adding furin, furin-like PCs or facilitated proteases. Then the anti-SARS-CoV-2 S1 or S2 antibody, depending on the tagged terminal, is applied to the wells. If the protein is cleaved, the S1 or S2 part of the protein recognized by the antibody against SARS-CoV-2 S1 or S2, respectively will be reduced and the detected signal will decrease accordingly. If the furin site cleavage is blocked by the generated fbAB, the signal will increase proportionally to the blockage intensity. Preferably, a peptide containing SARS-CoV-2 specific furin motif can be dual-tagged with the affinity moieties at both N- and C-terminals. For example, the peptide is tagged with polyhistidine at the N-terminal and biotin at the C-terminal and bound onto microplate wells through histidine/Ni-NTA at its N-terminal. The cleavage of the peptide at furin motif will remove the C-terminal part of the peptide after washing, which causes a decrease in signal generated by avidin/biotin binding after adding streptavidin-HRP conjugation solution. The blockage of the cleavage by the generated fbAB will block the reduction of the signals. The signal intensity is measured through an ELISA-like reaction by reading the absorbance or fluorescence in a spectrophotometer. The more the protein or peptide cleavage is blocked, the higher the signal that will be generated.

According to the method of the invention, blockage of SARS-CoV-2 specific furin motif by the generated fbAB can be further validated in the presence or absence of biological samples that may contain a mixed furin, furin-like PCs and facilitating proteases. The biological sample can be from various sources including fluids such as whole blood, serum, plasma, saliva, urine, milk, tears from the eye, ascites fluid, central spine fluid, peritoneal fluid and, amniotic fluid. In some embodiments, the sample could be a swab sample from the nasal cavity, oral cavity, nasopharyngeal, vagina and endocervix. In further embodiments, the sample can be solid tissues collected from various organs and can be cultured cells from laboratories. The liquid and solid samples can be collected by an appropriate methods or standard procedure, pre-treated to allow for immediate testing with the method of this invention. The sample may also be stored at an appropriate temperature for preserving the sample, for example stored at 4° C., −20° C. or −80° C. based on the sample types for a given length of time before test with the method of this invention.

It is unexpected that the generated fbAB has a high titer against the SARS-CoV-2 spike protein containing a specific furin site with strong binding capacity to such protein. It is also unexpected that the generated antibody can block the cleavage of the SARS-Cov-2 specific furin site caused not only by furin, but also by other serine proteases such as HAT and trypsin. Further, it is further unexpected that the generated fbAB is able to significantly reduce the binding of ACE2 to the SARS-CoV-2 spike protein.

The method of this invention for fbAB immunologically blocking furin cleavage site on the S1/S2 boundary of SARS-CoV-2 spike protein is further illustrated in the following examples:

### Example 1

The experiment was carried out to generate fbAB and to quantify the titer of the fbAB recognizing the antigen containing SARS-CoV-2 specific furin motif using the method of this invention.

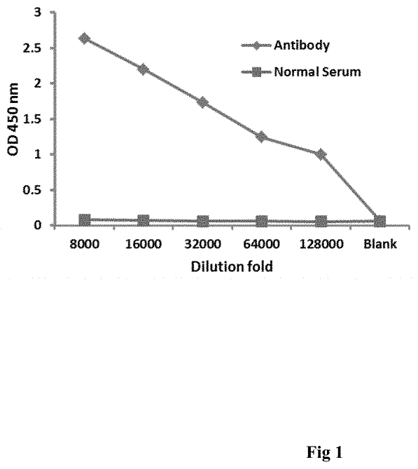

In this experiment, the peptide antigen containing ARS-Cov-2 specific furin motif (20 amino acids) was conjugated with KLH and 0.5 mg of conjugated antigen was injected into a rabbit. Then the rabbit was boosted with 0.25 mg of the antigen for 3 times in an interval of 2 weeks. The serum was collected and antibody was purified with protein A column. To test the titer of the generated fbAB, the antigen coated on the protein high binding polystyrene 8-well strip microplate at the concentration of 200 ng/well with 0.1M NaCO3 buffer. The strips were then incubated at 37° C. for 2 hours followed by blocking with 2% BSA for 1 hour. After wash with PBS-T for 3 times, the fbAB was diluted to the indicated concentrations with PBS-T buffer and was added into the wells for incubation for 1 hour. The wells were washed with PBS-T for 4 times. The anti-rabbit IgG conjugated with HRP was added into the wells at 1:2000 dilution and incubated for 30 min. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 1.

### Example 2

The experiment was carried out to determine the sensitivity of fbAB recognizing the SARS-CoV-2 protein containing S1/S2 boundary furin site using the method of this invention.

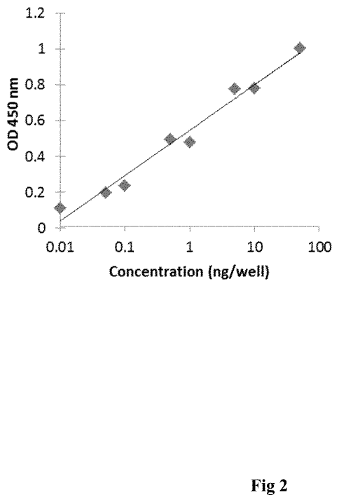

In this experiment, polystyrene 8-well strip microplate was coated with nickel (Ni-NTA). A SARS-CoV-2 protein containing S1/S2 boundary furin site is tagged with polyhistidine at N-terminal and bound onto microplate wells through histidine/Ni-NTA at its N-terminal at the different concentration. 50 ul of fbAB was added at 1:2000 to the wells and then incubated at room temperature for 1 hour. After wash with PBS-T for 3 times, 50 ul of the anti-rabbit IgG conjugated with HRP was added into the wells at the 1:2000 dilution and incubated for 30 min. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 2.

### Example 3

The experiment was carried out to examine the specificity of the fbAB recognizing the SARS-CoV-2 spike protein containing S1/S2 boundary furin site using the method of this invention.

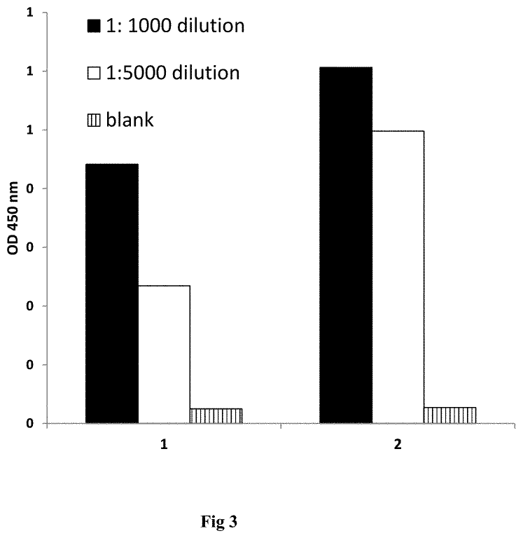

In this experiment, the SARS-CoV-2 protein containing S1/S2 boundary furin site, a peptide containing SARS-CoV-2 specific furin motif and SARS-CoV-2 S1 RBD protein lacking S1/S2 boundary furin site were tagged with polyhistidine. 10 ng of each protein/peptide was bound to the 8-well strip microplate coated with nickel (Ni-NTA). After wash with PBS-T for 2 times, 50 ul of fbAB was added at 1:1000 and 1:5000 to the wells and then incubated at room temperature for 1 hour. After wash with PBS-T for 3 times, 50 ul of the anti-rabbit IgG conjugated with HRP was added into the wells at the 1:2000 dilution and incubate for 30 min. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 3.

### Example 4

The experiment was carried out to examine the immunoprecipitation of the SARS-CoV-2 protein containing S1/S2 boundary furin site by the fbAB using the method of this invention.

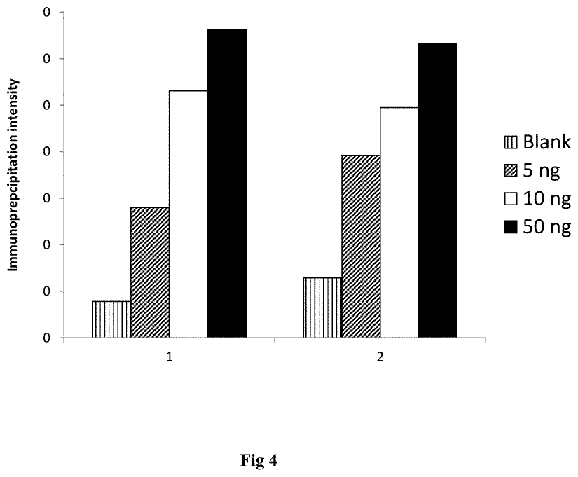

In this experiment, the fbAB was coated on the 8-well strip microplate at a concentration of 200 ng/well with 0.1M NaCO3 buffer. The strips were then incubated at 37° C. for 2 hours followed by blocking with 2% BSA for 1 hour. After washing with PBS-T for 3 times, the polyhistidine-tagged SARS-CoV-2 protein containing S1/S2 boundary furin site or polyhistidine tagged peptide containing SARS-CoV-2 specific furin motif was bound to fbAB coated wells at different concentrations by incubation at room temperature for 2 hours. After wash with PBS-T for 3 times, 50 ul of the Ni-NTA conjugated with HRP was added into the wells at 1:4000 dilution and incubate for 30 min. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 4.

### Experiment 5

The experiment was carried out to examine the fbAB blockage of the SARS-CoV-2 furin motif cleavage by furin using the method of this invention.

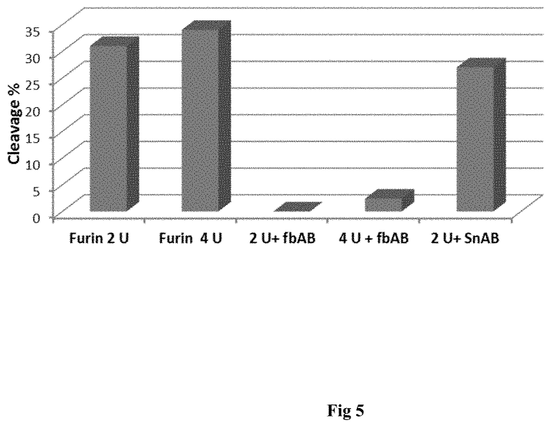

In this experiment, 10 ng of the peptide containing SARS-CoV-2 specific furin motif was tagged with polyhistidine at the N-terminal and biotin at the C-terminal and bound onto Ni-NTA coated 8-well strip microplate through histidine/Ni-NTA interaction. After incubation at room temperature for 1 hour, the wells were washed 3 times and fbAB was added into wells at the concentration of 200 ng/well and incubated at 37 C for 1 hour. A SARS-CoV-2 neutralization antibody (SnAB, Cusabio) was also added into the wells as a control. After washing 3 times with PBS-T, purified proprotein convertase furin (New England Biolabs) was diluted to an indicated concentration with PC assay buffer consisted of sodium phosphate, sodium chloride, and cesium chloride and was added into the wells for incubation for 25 min. The wells were washed with PBS-T for 4 times. The cleavage of the peptide at furin motif will remove the C-terminal part of the peptide after washing. 100 ul of streptavidin-HRP at 1:5000 dilution was then added into the wells for 15 min incubation. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 5.

### Experiment 6

The experiment was carried out to examine the fbAB blockage of the SARS-CoV-2 furin motif cleavage by facilitating protease trypsin using the method of this invention.

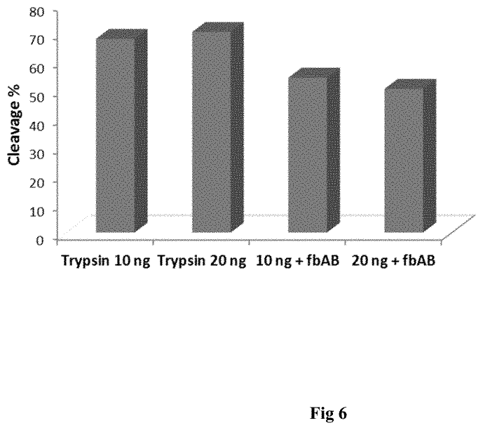

In this experiment, 10 ng of the peptide containing SARS-CoV-2 specific furin motif was tagged with polyhistidine at the N-terminal and biotin at the C-terminal and bound onto Ni-NTA coated 8-well strip microplate through histidine/Ni-NTA interaction. After incubation at room temperature for 1 hour, the wells were washed 3 times and fbAB was added into wells at the concentration of 200 ng/well and incubated at 37 C for 1 hour. After washing 3 times with PBS-T, serine protease trypsin (Sigma) was diluted to an indicated concentration with PC assay buffer consisted of sodium phosphate, sodium chloride, and cesium chloride and was added into the wells for incubation for 25 min. The wells were washed with PBS-T for 4 times. The cleavage of the peptide at furin motif will remove the C-terminal part of the peptide after washing. 100 ul of streptavidin-HRP at 1:5000 dilution was then added into the wells for 15 min incubation. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 6.

### Experiment 7

The experiment was carried out to examine the fbAB blockage of SARS-CoV-2 furin motif cleavage by human nasal swab sample using the method of this invention.

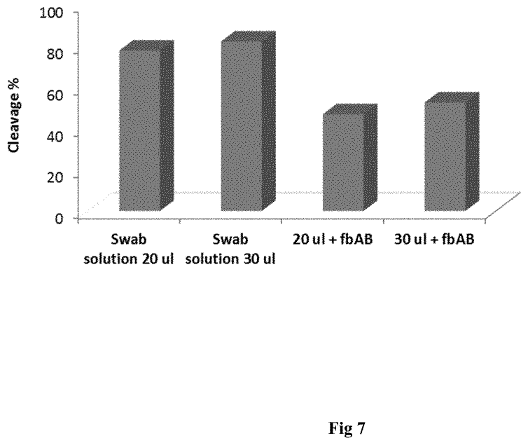

In this experiment, volunteer nasal swab samples were collected according to the standard procedure of swab samples. The collected swab samples were released into 300 μl of PC assay buffer (Epigentek) by rotating the swab in the buffer for 30 seconds and then used for the assay with 30 μl of the sample solution. 10 ng of the peptide containing SARS-CoV-2 specific furin motif was tagged with polyhistidine at the N-terminal and biotin at the C-terminal and bound onto Ni-NTA coated 8-well strip microplate through histidine/Ni-NTA interaction. After incubation at room temperature for 1 hour, the wells were washed 3 times and fbAB was added into wells at the concentration of 200 ng/well and incubated at 37 C for 1 hour. After washing 3 times with PBS-T, nasal swab sample solution was added into the wells for incubation for 25 min. The wells were washed with PBS-T for 4 times. The cleavage of the peptide at furin motif will remove the C-terminal part of the peptide after washing. 100 ul of streptavidin-HRP at 1:5000 dilution was then added into the wells for 15 min incubation. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 7.

### Example 8

The experiment was carried out to examine the effect of fbAB blocking the SARS-CoV-2 furin motif on ACE2 binding to SARS-CoV-2 spike protein using the method of this invention.

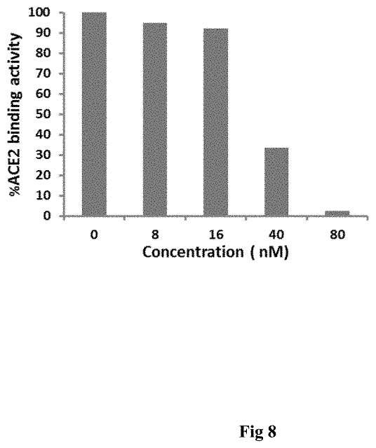

In this experiment, the un-tagged SARS-CoV-2 spike protein containing S1/S2 boundary furin site was coated on the protein high binding polystyrene 8-well strip microplate at concentration of 200 ng/well with 0.1M NaCO3 buffer. The strips were then incubated at 37° C. for 2 hours followed by blocking with 2% BSA for 1 hour. After wash with PBS-T for 3 times, fbAB was added into the wells at the concentration of 200 ng/well and incubated at 37 C for 1 hour. After washing 3 times with PBS-T, purified ACE2 tagged with polyhistidine (Sino Biological) was diluted to an indicated concentration with PBS buffer and was added into the wells for incubation for 1 hour. After washing 3 times, 50 ul of the Ni-NTA conjugated with HRP was added into the wells at the 1:4000 ratio and incubate for 30 min. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm. The results are shown in the FIG. 8.

What is claimed is:

1. An antibody or antigen binding fragment therefor that specifically binds to S1/S2 boundary furin site contained in the SARS-CoV-2 spike protein and blocks the enzyme cleavage of SARS-CoV-2 protein S1/S2 boundary furin site in a biological body fluid, tissues and cells.

2. The antibody of claim 1, where in the antibody recognizes an epitope comprising SARS-CoV-2 spike amino acid ^{A}672-^{S}691.

3. The antibody of claim 1, where in the antibody binds to an epitope within the amino acid ^{A}672-^{S}691 of SARS-CoV-2 spike protein.

4. The antibody of claim 1, wherein the antibody is an IgG antibody.

5. The antibody of claim 1, wherein the antibody is a polyclonal antibody, a monoclonal antibody a recombinant antibody, a human antibody, a humanized antibody, or a chimeric antibody thereof.

6. The antibody of claim 1, wherein said S1/S2 boundary furin site contains furin cleavage motif containing at least 3 arginine amino acids.

7. The antibody of claim 1, wherein said enzyme is a serine protease selected from a serine protease group of furin, PC1/3, PC2, PACE4, PC5/6A, PC7, SKI-1/S1P and PC9; human air trypsin (HAT), TMPRSS2, TMPRSS4, TMPRSS11A, matriptase; plasmin, thrombin, and tissue activating plasminogen.

8. The antibody of claim 1, wherein said biological body fluid is human blood.

9. The antibody of claim 1, wherein said tissues and cells are human tissues and cells.

10. The antibody of claim 1, wherein the antibody is capable of neutralizing SARS-CoV-2 by blocking furin site cleavage-caused reduction of ACE2 binding to SARS-CoV-2 spike protein.

11. A method for reducing pathogenicity and transmission of COVID-19 by blocking furin cleavage of SRAS-CoV-2 spike protein S1/S2 boundary furin site comprising administering to a subject in need thereof an effective amount of the antibody of claim 1.

12. A method of claim 11, wherein said S1/S2 boundary furin site within the amino acid ^{A}672 ^{S}691 of SARS-CoV-2 spike protein.

13. A method of claim 11, wherein said subject suffer from COVID-19.

14. A pharmaceutical composition comprising the antibody of claim 1 and a pharmaceutically acceptable carrier.
